SEQUENCE LISTING (15 sequences)

SEQ ID NO: 1 - RNA, 22 nt, Homo sapiens
uagcagcacauaaugguuugug

SEQ ID NO: 2 - RNA, 22 nt, Homo sapiens
uagcagcacaucaugguuuaca

SEQ ID NO: 3 - RNA, 23 nt, Homo sapiens
agcuacauugucugcuggguuuc

SEQ ID NO: 4 - RNA, 22 nt, Homo sapiens
aaaccguuaccauuacugaguu

SEQ ID NO: 5 - RNA, 17 nt, Homo sapiens
ucucgcuggggccucca

SEQ ID NO: 6 - RNA, 19 nt, Homo sapiens
aauggauuuuuggagcagg

SEQ ID NO: 7 - RNA, 18 nt, Homo sapiens
gcaugggugguucagugg

SEQ ID NO: 8 - RNA, 20 nt, Homo sapiens
ccccagggcgacgcggcggg

SEQ ID NO: 9 - RNA, 23 nt, Homo sapiens
ugguuguaguccgugcgagaaua

SEQ ID NO: 10 - RNA, 23 nt, Homo sapiens
ugagugugugugugugagugugu

SEQ ID NO: 11 - RNA, 22 nt, Homo sapiens
ggggcuggggccggggccgagc

SEQ ID NO: 12 - RNA, 23 nt, Homo sapiens
aacauucaacgcugucggugagu

SEQ ID NO: 13 - RNA, 23 nt, Homo sapiens
aacauucauugcugucggugggu

SEQ ID NO: 14 - RNA, 22 nt, Homo sapiens
uagcagcacguaaauauuggcg

SEQ ID NO: 15 - RNA, 25 nt, Homo sapiens
agggaucgcgggcggguggcggccu

(19) United States
(12) Patent Application Publication
(10) Pub. No.: US 2014/0194467 A1
(43) Pub. Date: Jul. 10, 2014

(54) PLASMA CELL DISORDERS

(71) Applicants: Sarah Faith Newbury, Brighton, East Sussex (GB); Timothy James Telfer Chevassut, Brighton, East Sussex (GB); Christopher Iain Jones, Brighton, East Sussex (GB); Maria Vasilyevna Zabolotskaya, Brighton, East Sussex (GB); Helen Jayne Susan Stewart, Brighton, East Sussex (GB)

(72) Inventors: Sarah Faith Newbury, Brighton, East Sussex (GB); Timothy James Telfer Chevassut, Brighton, East Sussex (GB); Christopher Iain Jones, Brighton, East Sussex (GB); Maria Vasilyevna Zabolotskaya, Brighton, East Sussex (GB); Helen Jayne Susan Stewart, Brighton, East Sussex (GB)

(73) Assignee: THE UNIVERSITY OF SUSSEX, Brighton, East Sussex (GB)

(21) Appl. No.: 13/993,834

(22) PCT Filed: Dec. 13, 2011
(86) PCT No.: PCT/GB2011/052464

§ 371 Date: Sep. 12, 2013

(30) Foreign Application Priority Data
Dec. 13, 2010 (GB) 1021107.6

(51) Int. Cl. C12Q 1/68; A61K 31/454

(52) U.S. Cl. CPC C12Q 1/6886; A61K 31/454
USPC 514/323; 435/6.12

(57) ABSTRACT

The invention relates to novel biological markers for plasma cell disorders, such as multiple myeloma, and in particular to the use of microRNAs as diagnostic and prognostic markers in assays for detecting such disorders. The invention also relates to methods of determining the efficacy of treating a plasma cell disorder with a therapeutic agent, and kits for carrying 5 out the assays and methods. The assays are qualitative and/or quantitative, and are adaptable to large-scale screening and clinical trials.

The present invention relates to novel biological markers for plasma cell disorders, such as multiple myeloma, and in particular to the use of microRNAs as diagnostic and prognostic markers in assays for detecting such disorders. The invention also relates to methods of determining the efficacy of treating a plasma cell disorder with a therapeutic agent, and kits for carrying out the assays and methods. The assays are qualitative and/or quantitative, and are adaptable to large-scale screening and clinical trials.

Plasma cells are white blood cells produced from B lymphocytes, and create and release antibodies to fight infection. Plasma cells do not normally circulate in the blood; they are usually located only in bone marrow, lymph nodes and areas where an immune response takes place. Plasma cell disorders occur when one plasma cell clone, which is capable of producing a monoclonal antibody, divides and multiples uncontrollably. These abnormal plasma cells eventually begin to “crowd out” other plasma cells and, as a result, the body's ability to fight viruses, bacteria and other infections decreases dramatically. The abnormal plasma cells may eventually invade the surrounding bone, resulting in bone lesions, abnormal amounts of calcium in the blood and resulting organ damage, such as kidney failure.

Common plasma cell disorders include amyloidosis, Waldenstrom's macroglobulinemia osteosclerotic myeloma (POEMS syndrome), Monoclonal gammopathy of unknown significance (MGUS) or Plasma cell myeloma. Amyloidosis occurs when antibodies or protein fragments accumulate in organs of the body with the build-up of antibody or protein fragments resulting in a reduced ability to fight infection. Abnormal plasma cells multiply uncontrollably and group together at multiple sites in the bone marrow, crowding out healthy blood cells. Waldenstrom's macroglobulinemia, on the other hand, is a malignant blood cancer characterized by a high level of IgM antibodies in the blood and the bone marrow.

Multiple myeloma, also termed myeloma, is a malignant disorder of bone marrow plasma cells that accounts for approximately 10% of all haematological cancers and 1% of all cancers. The annual incidence of multiple myeloma age-adjusted to the 2006 UK population is 6.6 per 100,000, therefore affecting more that 3000 new individuals per year.

The most common symptoms on presentation are fatigue, bone pain and recurrent infections, with the median survival after diagnosis being approximately three years after diagnosis. Although myeloma is presently incurable, recent advances in treatment, including the use of thalidomide and new drugs such as bortezomib, as well as autologous stem cell transplantation, can improve life expectancy.

Myeloma usually progresses from an asymptomatic pre-cancerous stage of clonal plasma cell proliferation termed “monoclonal gammopathy of undetermined significance” (MGUS). MGUS is present in more that 3% of the population above the age of 50 and progresses to myeloma or a related malignancy at a rate of ˜1% per year. The sequence of events responsible for malignant transformation of MGUS to multiple myeloma is, at present, not well understood. Since no single factor can identify those patients with MGUS likely to progress to myeloma, patients need to be monitored at regular intervals. Therefore, a diagnostic method to identify those patients who are most at risk of cancer progression would be of immense benefit to patients and would raise the possibility of early treatment and improved prognosis. Hence, there is need to provide a reliable biological marker for predicting which patients will progress to multiple myeloma.

According to a first aspect of the present invention, there is provided a method for diagnosing a subject suffering from a plasma cell disorder, or a pre-disposition thereto, or for providing a prognosis of the subject's condition, the method comprising analysing the concentration of one or more type of microRNA molecule in a bodily sample from a test subject and comparing this concentration with a reference for the concentration of the one or more type of microRNA molecule in an individual who does not suffer from a plasma cell disorder, wherein a difference in the concentration of the one or more type of microRNA molecule in the bodily sample from the test subject compared to the reference suggests that the subject is suffering from a plasma cell disorder, or has a pre-disposition thereto, or provides a negative prognosis of the subject's condition.

According to a second aspect of the present invention, there is provided a method for determining the efficacy of treating a subject suffering from a plasma cell disorder with a therapeutic agent, the method comprising analysing the concentration of one or more type of microRNA molecule in a bodily sample from a test subject and comparing this concentration with a reference for the concentration of the one or more type of microRNA molecule in an individual who does not suffer from a plasma cell disorder, wherein a difference in the concentration of the one or more type of microRNA molecule in the bodily sample compared to the reference is indicative of the efficacy of treating the test subject with the therapeutic agent.

According to a third aspect of the invention, there is provided a kit for diagnosing a subject suffering from a plasma cell disorder, or a pre-disposition thereto, or for providing a prognosis of the subject's condition, the kit comprising:
(i) means for determining the concentration of one or more type of micro RNA molecule in a sample from a test subject; and
(ii) a reference for the concentration of the one or more type of microRNA molecule in a sample from an individual who does not suffer from a plasma cell disorder,
   wherein the kit is used to identify a difference in the concentration of the one or more type of microRNA molecule in the sample from the test subject compared to the reference concentration, thereby suggesting that the test subject suffers from a plasma cell disorder, or has a pre-disposition thereto, or providing a negative prognosis of the subject's condition.

According to a fourth aspect of the invention, there is provided a kit for determining the efficacy of treating a subject suffering from a plasma cell disorder with a therapeutic agent, the kit comprising:
(i) means for determining the concentration of one or more type of microRNA molecule in a sample from a test subject; and
(ii) a reference for the concentration of the one or more type of microRNA molecule in a sample from an individual who does not suffer from a plasma cell disorder,
   wherein the kit is used to identify a difference in the concentration of the one or more type of microRNA molecule in the sample from the test subject compared to the reference concentration, the difference in concentration being indicative of the efficacy of treating the test subject with the therapeutic agent.

According to a fifth aspect of the invention, there is provided a method of treating an individual suffering from a plasma cell disorder, said method comprising the steps of:
(i) determining the concentration of one or more microRNA molecule in a sample having been obtained from a test subject, wherein a difference in theconcentration of the one or more type of microRNA molecule in the bodily sample from the test subject compared to the concentration of the one or more type of microRNA molecule in a sample from an individual who does not suffer from a plasma cell disorder, suggests that the test subject suffers from a plasma cell disorder, or is pre-disposed thereto, or has a negative prognosis; and
(ii) administering, to the test subject, a therapeutic agent that prevents, reduces or delays progression of the plasma cell disorder.

An important feature of any useful biomarker used in disease diagnosis and prognosis is that it exhibits high sensitivity and specificity for a given disease. Firstly, as explained in the examples, the inventors have surprisingly demonstrated that microRNA molecules (miRNAs) can survive in the peripheral blood. Secondly, they have found that these miRNAs can serve as robust biomarkers for plasma cell disorders, such as myeloma, and can therefore be used for the detection of cancerous and pre-cancerous states, and disease prognosis. In addition, the inventors have shown that using miRNAs as biomarkers for plasma cell disorders employs an assay which is simple, reproducible and inexpensive, and with minimal inconvenience to the patient.

Up until recently, the possibility of using miRNAs as a routine, non-invasive test for plasma cell disorders, such as myeloma and “monoclonal gammopathy of undetermined significance” (MGUS) patients, has not been possible because bone marrow samples need to be taken, a procedure which is painful to the patient and requires a skilled practitioner. However, the inventors have surprisingly shown that miRNAs can be detected in blood plasma and serum, opening the way to a novel, non-invasive diagnostic test. Furthermore, advantageously, miRNAs are stable in plasma at room temperature (for at least 24 hours) and when subjected to freeze-thaw cycles. Coupled with this, and the data described in the examples, it is clearly possible to use miRNAs derived from blood samples as a minimally invasive diagnostic tool for patients suffering from plasma cell disorders.

The method according to the first aspect is useful for enabling a clinician to make decisions with regards to the best course of treatment for a subject who is currently or who may suffer from a plasma cell disorder in the future, such as myeloma. It is preferred that the method of the first aspect is useful for enabling a clinician to decide how to treat a subject who is suffering from a plasma cell disorder. In addition, the methods of the first and second aspects are useful for monitoring the efficacy of a putative treatment for a plasma cell disorder, for example thalidomide or bortezomib when treating myeloma. Hence, the kits according to the third and fourth aspects are useful for providing a prognosis of the subject's condition, such that the clinician can carry out the treatment according to the fifth aspect. The kit of the third aspect may be used to monitor the efficacy of a putative treatment for plasma cell disorders. The methods and the kits are therefore very useful for guiding a plasma cell disorder treatment regime for the clinician, and to monitor the efficacy of such a treatment regime. The clinician may use the kits of the invention in conjunction with existing diagnostic tests to improve the accuracy of diagnosis.

The methods and kits of the invention may be used to predict the risk of progression in a pre-cancerous and malignant subject. The plasma cell disorder may for example be amyloidosis, Waldenstrom's macroglobulinemia, osteosclerotic myeloma (POEMS syndrome), Monoclonal gammopathy of unknown significance (MGUS) or Plasma cell myeloma. Preferably, the disorder is plasma cell myeloma, i.e. multiple myeloma.

MicroRNA molecules are non-coding, post-transcriptional regulators that normally bind to complementary sequences in the 3′ untranslated regions (3′ UTRs) of target messenger RNA transcripts (mRNAs), usually resulting in gene silencing. miRNAs are short ribonucleic acid (RNA) molecules, on average only about 22 nucleotides long. Thus, the miRNA detected in the methods and kits of the invention may be about 15 to 30 nucleotides long, or about 18 to 25 nucleotides long, or about 21 to 23 nucleotides long.

The methods and kits of the invention may comprise analysing the concentration of any of the known miRNAs, and which can be found on the miRBase website (http://www.mirbase.org/). The miRBase (release 18) currently includes 1587 mature human miRNAs, all of which are processed from longer precursors and differ from each other in nucleotide sequence. The current understanding is that each miRNA is expressed in one or more human tissues and binds to one or more target RNA sequences expressed in particular tissues. The binding of this single miRNA, by itself or in combination with other miRNAs and proteins to a particular mRNA, leads to down-regulation of gene expression, usually by degradation of the target mRNA or repression of protein translation.

It is preferred that the methods and kits may comprise analysing the concentration of one or more type of microRNA molecule selected from the group of miRNA molecules consisting of miRNA-15a, miRNA-15b, miRNA-16, miRNA-221, miRNA-451, miRNA-638, miRNA-720, miRNA-1246, miRNA-1308, miRNA-1915, miRNA-1974, miRNA-574-5p and miRNA-762.

Hence, in a sixth aspect, there is provided use of one or more type of microRNA, as a biomarker for diagnosing a subject suffering from a plasma cell disorder, or a pre-disposition thereto, or for providing a prognosis of the subject's condition, wherein the one or more type of microRNA molecule is selected from the group of miRNA molecules consisting of miRNA-15a, miRNA-15b, miRNA-16, miRNA-221, miRNA-451, miRNA-638, miRNA-720, miRNA-1246, miRNA-1308, miRNA-1915, miRNA-1974, miRNA-574-5p and miRNA-762.

In one embodiment, the miRNA being detected is miRNA-15a. The sequence of miRNA-15a is 22 nucleotides long, and is referred to herein as SEQ ID No.1, as follows:

SEQ ID No. 1
5′-UAGCAGCACAUAAUGGUUUGUG-3′

Therefore, the miRNA may comprise a nucleotide sequence substantially as set out in SEQ ID No.1, or the complementary sequence thereof, or a variant or fragment thereof.

In another embodiment, the miRNA being detected is miRNA-15b. The sequence of miRNA-15b is 22 nucleotides long, and is referred to herein as SEQ ID No.2, as follows:

SEQ ID No. 2
5′-UAGCAGCACAUCAUGGUUUACA-3′

Therefore, the miRNA may comprise a nucleotide sequence substantially as set out in SEQ ID No.2, or the complementary sequence thereof, or a variant or fragment thereof.

In another embodiment, the miRNA being detected is miRNA-16. The sequence of miRNA-16 is 22 nucleotides long, and is referred to herein as SEQ ID No.3, as follows:

SEQ ID No. 3
5′-UAGCAGCACGUAAAUAUUGGCG-3′

Therefore, the miRNA may comprise a nucleotide sequence substantially as set out in SEQ ID No.3, or the complementary sequence thereof, or a variant or fragment thereof.

In one embodiment, the miRNA being detected is miRNA-221. The sequence of miRNA-221 is 23 nucleotides long, and is referred to herein as SEQ ID No.4, as follows:

SEQ ID No. 4
5′-AGCUACAUUGUCUGCUGGGUUUC-3′

Therefore, the miRNA may comprise a nucleotide sequence substantially as set out in SEQ ID No.4, or the complementary sequence thereof, or a variant, or fragment thereof.

In one embodiment, the miRNA being detected is miRNA-451. The sequence of miRNA-451 is 22 nucleotides long, and is referred to herein as SEQ ID No.5, as follows:

SEQ ID No. 5
5′-AAACCGUUACCAUUACUGAGUU-3′

Therefore, the miRNA may comprise a nucleotide sequence substantially as set out in SEQ ID No.5, or the complementary sequence thereof, or a variant, or fragment thereof.

In one embodiment, the miRNA being detected is miRNA-638. The sequence of miRNA-638 is 25 nucleotides long, and is referred to herein as SEQ ID No.6, as follows:

SEQ ID No. 6
5′-AGGGAUCGCGGGCGGGUGGCGGCCU-3′

Therefore, the miRNA may comprise a nucleotide sequence substantially as set out in SEQ ID No.6, or the complementary sequence thereof, or a variant, or fragment thereof.

In one embodiment, the miRNA being detected is miRNA-720. The sequence of miRNA-720 is 17 nucleotides long, and is referred to herein as SEQ ID No.7, as follows:

SEQ ID No. 7
5′-UCUCGCUGGGGCCUCCA-3′

Therefore, the miRNA may comprise a nucleotide sequence substantially as set out in SEQ ID No.7, or the complementary sequence thereof, or a variant, or fragment thereof.

In one embodiment, the miRNA being detected is miRNA-1246. The sequence of miRNA-1246 is 19 nucleotides long, and is referred to herein as SEQ ID No.8, as follows:

SEQ ID No. 8
5′-AAUGGAUUUUUGGAGCAGG-3′

Therefore, the miRNA may comprise a nucleotide sequence substantially as set out in SEQ ID No.8, or the complementary sequence thereof, or a variant, or fragment thereof.

In one embodiment, the miRNA being detected is miRNA-1308. The sequence of miRNA-1308 is 18 nucleotides long, and is referred to herein as SEQ ID No.9, as follows:

SEQ ID No. 9
5′-GCAUGGGUGGUUCAGUGG-3′

Therefore, the miRNA may comprise a nucleotide sequence substantially as set out in SEQ ID No.9, or the complementary sequence thereof, or a variant, or fragment thereof.

In one embodiment, the miRNA being detected is miRNA-1915. The sequence of miRNA-1915 is 20 nucleotides long, and is referred to herein as SEQ ID No.10, as follows:

SEQ ID No. 10
5′-CCCCAGGGCGACGCGGCGGG-3′

Therefore, the miRNA may comprise a nucleotide sequence substantially as set out in SEQ ID No.10, or the complementary sequence thereof, or a variant, or fragment thereof.

In one embodiment, the miRNA being detected is miRNA-1974. The sequence of miRNA-1974 is 20 nucleotides long, and is referred to herein as SEQ ID No.11, as follows:

SEQ ID No. 11
5′-UGGUUGUAGUCCGUGCGAGAAUA-3′

Therefore, the miRNA may comprise a nucleotide sequence substantially as set out in SEQ ID No.11, or the complementary sequence thereof, or a variant, or fragment thereof.

In one embodiment, the miRNA being detected is miRNA-574-5p. The sequence of miRNA-574-5p is 23 nucleotides long, and is referred to herein as SEQ ID No.12, as follows:

SEQ ID No. 12
5′-UGAGUGUGUGUGUGUGAGUGUGU-3′

Therefore, the miRNA may comprise a nucleotide sequence substantially as set out in SEQ ID No.12, or the complementary sequence thereof, or a variant, or fragment thereof.

In one embodiment, the miRNA being detected is miRNA-762. The sequence of miRNA-762 is 22 nucleotides long, and is referred to herein as SEQ ID No.13, as follows:

SEQ ID No. 13
5′-GGGGCUGGGGCCGGGGCCGAGC-3′

Therefore, the miRNA may comprise a nucleotide sequence substantially as set out in SEQ ID No.13, or the complementary sequence thereof, or a variant, or fragment thereof.

The methods and kits of the invention may comprise determining the concentration of one or more type of microRNA molecule selected from the group of miRNA molecules comprising a nucleotide sequence substantially as set out in any or SEQ ID No.1 to 13, or any of the complementary sequences thereof, or variants or fragments thereof. The pattern of expression, where miRNAs 15a, 15b, 16, 221, 638, 720, 1246, 451, 1308 and/or 1915 are found at either significantly higher or significantly lower levels in a bodily sample compared to a test subject (e.g. plasma or serum from peripheral blood) may be termed the “miRNA signature”.

The complementary sequence of SEQ ID No's 1-13 (often termed miRNA*, for example miRNA-15a*, miRNA-15b*, miRNA-16*, miRNA-221*, miRNA-451*, miRNA-638*, miRNA-720*, miRNA-1246*, miRNA-1308*, miRNA-1915*, miRNA-1974*, miRNA-574-5p* or miRNA-762*) may also be detected. These complementary sequences have also been shown to have activity, and therefore also have utility as a biomarker for plasma cell disorders.

Variants and fragments of any of the miRNA molecules that may be detected may include truncations or additions of nucleotides of the miRNA molecule, for example SEQ ID No.1-13. A truncation may comprise the miRNA molecule having been reduced in size by the removal of at least one nucleotide from the 5′ and/or 3′ end of the miRNA, or by deletion of one of more nucleotides from within the core or centre of the miRNA. The truncation may comprise deletion of at least 2, 3, 4 or 5 nucleotides from the miRNA molecule. An addition may comprise the miRNA molecule having been increased in size by the addition of at least one nucleotide to the 5′ and/or 3′ end of the miRNA, or by the introduction of one of more nucleotides into the core or centre of the miRNA. The addition may comprise addition of at least 2, 3, 4, 5, or up to 10 nucleotides to the miRNA molecule.

The concentration of the at least one type of miRNA molecule may act as a diagnostic and/or prognostic marker for a plasma cell disorder, for example myeloma. In addition, the miRNA may also act as a diagnostic and/or prognostic marker for benign diseases. For example, the disorder may be benign following chemotherapeutic treatment regimes. The inventors investigated the expression levels of a large number of miRNA molecules in myeloma patients, and were surprised to observe that a number of miRNAs (i.e. miRNAs 15a, 15b, 16, 221, 638, 720, 1246 and/or 1915) were over-expressed in myeloma patients compared to the controls. Similarly, the inventors have also found that the concentration of a number of miRNA molecules (i.e. miRNAs 451 and/or 1308) decreased when compared to the controls, and that these also form a miRNA signature. The inventors therefore realised that these miRNA molecules, which together form a miRNA signature, represents a useful and robust physiological marker of myeloma and other plasma cell disorders in patients. Furthermore, surprisingly the inventors observed that miRNA-638, miRNA-720, miRNA-1246, miRNA-1308 and miRNA-1915 exhibited different expression patterns in myeloma, MGUS and control patients, and so these miRNAs are preferred. Accordingly, each of these biomarkers can be robustly used for prognostic and diagnostic purposes.

The inventors have established that circulating levels of miRNAs in a test subject is highly suggestive of the whether the subject either has, or is pre-disposed to developing, a plasma cell disorder, and is sufficiently sensitive to detect the disorder at an early stage. In addition, assaying for the miRNA can differentiate between benign or malignant conditions. Furthermore, using the invention, it is possible to monitor whether a subject's condition relapses following a treatment regime. Accordingly, the methods of the first and second aspects of the invention, in which the concentration of the miRNA molecule is measured, and compared to a control concentration, provides a very reliable prognostic marker for monitoring conditions, both before and after treatment. Accordingly, assaying for miRNA molecules is a substantial improvement over assaying for other markers, because it is more sensitive and also provides enhanced specificity. In addition, assaying for miRNA molecules also provides far more information to the clinician, and will help stratify the disease.

It will be appreciated that detecting one particular type of miRNA molecule may be of use by itself as a biomarker for a plasma cell disorder. Further, detecting more than one type of miRNA molecule, may provide a more robust diagnosis or prognosis of the disease. In addition, the biomarker may also be used in combination with an assay of another biological marker indicative of plasma cell disorders, for example detection of aberrant monoclonal paraprotein and/or serum free light chain. For example, paraprotein or light chain may be detected by serum or urine electrophoresis. Hence, assaying for one or more miRNA molecules may be used to complement the use of another marker to provide even more information to the clinician.

The subject may be any animal of veterinary interest, for instance, a cat, dog, horse etc. However, it is preferred that the subject is a mammal, such as a human, either male or female.

Preferably, a sample is taken from the subject, and the concentration of the one or more type of miRNA molecule may be measured. The kit of the third or fourth aspect may comprise sample extraction means for obtaining the sample from the test subject. The sample extraction means may comprise a needle or syringe or the like.

It has been demonstrated that miRNAs occur in body and organ fluids, such as cerebrospinal fluid or follicular fluid. However, the sample may be any bodily sample into which miRNA molecules are secreted, e.g. it may be lymph or interstitial fluid. The sample may be a urine sample. However, the inventors were very surprised to observe that miRNAs survive in the blood. Therefore, it is preferred that the miRNA molecule is measured or assayed in a blood sample. The blood sample may be venous or arterial.

The kit may comprise a sample collection container for receiving the extracted sample. Blood samples may be assayed for miRNA molecule levels immediately. Alternatively, the blood may be stored at low temperatures, for example in a fridge or even frozen before the miRNA assay is conducted. Measurement of miRNA may be made on whole blood.

However, the blood may be further processed before the assay is performed. For instance, an anticoagulant, such as citrate (such as sodium citrate), hirudin, heparin, PPACK, or sodium fluoride may be added. Thus, the sample collection container may contain an anticoagulant in order to prevent the blood sample from clotting. Alternatively, the blood sample may be centrifuged or filtered to prepare a plasma or serum fraction, which may be used for analysis. Hence, it is preferred that the miRNA is analysed or assayed in a blood plasma or a blood serum sample. It is preferred that miRNA concentration is measured in vitro from a blood serum sample or a plasma sample taken from the subject.

It will also be appreciated that “fresh” bodily samples may be analysed immediately after they have been taken from a subject. Alternatively, the samples may be frozen and stored. The sample may then be de-frosted and analysed at a later date.

As described in the examples, the inventors monitored the concentration of various miRNAs in numerous patients who suffered from myeloma, and compared them to the concentration of the same miRNAs in individuals who did not suffer from myeloma. They demonstrated that there was a statistically significant increase or decrease in the concentration of certain miRNA molecules described herein in the patients suffering from myeloma. Thus, the difference in concentration may be an increase or a decrease compared to the reference. It will be appreciated that the concentration of a certain miRNA molecule in myeloma patients is highly dependent on a number of factors, for example how far the disease has progressed, and the age and gender of the subject. It will also be appreciated that the concentration of miRNAs in individuals who do not suffer from a plasma cell disorder may fluctuate to some degree, but that on average over a given period of time, the concentration tends to be substantially constant. In addition, it should be appreciated that the concentration of miRNA in one group of individuals who do not suffer from a plasma cell disorder may be different to the concentration of those miRNAs in another group of individuals who do not suffer from the disease. However, the skilled technician will know how to determine the average concentration of certain miRNAs in individuals who do not suffer from a plasma cell disorder, and this is referred to as the ‘normal’ concentration of miRNA. The normal concentration corresponds to the reference values discussed above in the first to fifth aspects.

The miRNAs may be extracted from the bodily sample by a variety of techniques. Briefly, these may comprise addition of a protein denaturant (such as Trizol or guanidine thiocyanate) to the sample, centrifugation to remove protein debris, addition of DNaseI to remove DNA, and extraction of RNA using a suitable column. RNA samples may be further concentrated by ethanol/isopropanol precipitation and/or centrifugal concentration. At present, the preferred extraction kit used by the inventors is supplied by Ambion, but other extraction kits could be used, depending on availability and/or suitability in subsequent downstream reactions.

PCR may be used to amplify the one or more type of miRNA molecule. The PCR technology may be selected from the group consisting of real-time PCR, reverse transcriptase PCR, multiplex PCR or molecular beacon PCR. It will be appreciated that

PCR involves the use of two primers which are substantially complementary to the miRNA molecule being assayed in the sample. The kits according to the third and fourth aspects comprise means for determining the concentration of one or more type of miRNA molecule in a sample from a test subject. The kit may comprise a container in which the means for determining the concentration of one or more type of miRNA molecule in a sample from a test subject may be contained. The kit may also comprise instructions for use.

Thus, the kits may comprise detection means for determining the concentration of the one or more type of miRNA in the sample once this has been obtained from the subject. For example, the detection means may comprise one or more primer, for use in a PCR method for amplifying the miRNA. In one embodiment, detection of the one or more type of miRNA molecule may be achieved by TaqMan quantitative RT-PCR using primer and probe sets specific for particular human miRNAs, as described on the Applied Biosystems website (http://www.appliedbiosystems.com/absite/us/en/home.html). This method makes use of looped Reverse Transcriptase primers to generate the cDNA and then forward and reverse primers for the PCR amplification. Quantification is achieved by use of a fluorescently labelled probe, situated between the two primers, where fluorescence is activated upon the PCR reaction (for method see http://www.appliedbiosystems.com/absite/us/en/home.html).

In another embodiment, detection may be achieved using an Exiqon microRNA detection kit. (http://www.exiqon.com/ls). However, other PCR-based and microarray-based detection methods are also applicable to this invention. The primers may comprise at least partial sequence identity with the miRNA molecule being detected, for example miRNA-15a, miRNA-15b, miRNA-16, mi-RNA-221, miRNA-451, miRNA-638, miRNA-720, miRNA-1246, miRNA-1308, miRNA-1915, miRNA-1974, miRNA-574-5p or miRNA-762. In another embodiment, the Reverse Transcriptase and PCR reactions may comprise the procedure as set out in Example 1.

The reference values may be obtained by assaying a statistically significant number of control samples (i.e. samples from subjects who do not suffer from a plasma cell disorder). Accordingly, the reference (ii) according to the kits of the third and fourth aspects of the invention may be a control sample (for assaying).

The kit may comprise a positive control (preferably provided in a container), which corresponds to total RNA extracted from a sample (e.g. the plasma) of a subject having a plasma cell disorder (e.g. myeloma) where it has been established that the relevant miRNAs (for example, miRNA-15a, miRNA-15b, miRNA-16, miRNA-221, miRNA-451, miRNA-638, miRNA-720, miRNA-1246, miRNA-1308, miRNA-1915, miRNA-1974, miRNA-574-5p and/or miRNA-762) are present at statistically higher or lower levels than normal controls (i.e. a normal healthy subject without a plasma cell disorder, such as myeloma). Hence, the positive control miRNA may comprise a nucleotide sequence substantially as set out in SEQ ID No.1-13, or the complementary sequence thereof, or a variant, or fragment thereof.

The kit may comprise a negative control (preferably provided in a container), which corresponds to total RNA extracted from a sample (e.g. the plasma) of a normal, healthy subject without a plasma cell disorder (e.g. myeloma), where it has previously been established that the above miRNAs are not detectable. By way of example only, the miRNAs 181a and 181b were found to be present at levels that were not detectably higher than normal, healthy controls, though it will be appreciated that there are many other miRNAs which would fall into this category. Therefore, for example miRNAs 181a and 181b may be used as negative controls.

The sequence of miRNA-181a is 23 nucleotides long, and is referred to herein as SEQ ID No. 14, as follows:

SEQ ID No. 14
5′-AACAUUCAACGCUGUCGGUGAGU-3′

The sequence of miRNA-181b is 23 nucleotides long, and is referred to herein as SEQ ID No. 15, as follows:

SEQ ID No. 15
5′-AACAUUCAUUGCUGUCGGUGGGU-3′

Hence, the negative control miRNA may comprise a nucleotide sequence substantially as set out in SEQ ID No.14 or 15, or the complementary sequence thereof, or a variant or fragment thereof.

In a preferred embodiment, the kit may comprise the reference, a positive control and a negative control. The kit will also comprise further controls, as necessary, such as “spike-in” controls to provide a reference for concentration, and further positive controls for each of the “signature” microRNAs.

Hence, by way of example only, the blood plasma concentration of the signature miRNA in normal, healthy individuals not suffering from a plasma cell disorder may not be detectable, whereas the concentration of certain signature miRNAs in a patient with a plasma cell disorder (e.g. myeloma) may be at least 5-, 10, 15- or 20-fold higher. Also, by way of example, the decrease in concentration of certain signature miRNAs in a plasma cell disorder may be at least 5-, 10, 15- or 20-fold lower than a healthy patient.

The inventors noted that the concentration of certain miRNAs (for example, miRNAs 15a, 15b, 16, 221, 638, 720, 1246 and/or 1915) in the test individuals was statistically more than the reference concentration (as calculated using the method described in the Example). This may be referred to herein as the ‘increased’ concentration of the one or more miRNA. Conversely, the inventors also noted that the concentration of certain other miRNAs (for example, miRNAs 451 and/or 1308) in the test individuals was statistically less than the reference concentration (as calculated using the method described in the Example). This may be referred to herein as the ‘decreased’ concentration of the one or more miRNA.

The skilled technician will appreciate how to measure the concentrations of miRNAs in a statistically significant number of control individuals, and the concentration of miRNA in the test subject, and then use these respective figures to determine whether the test subject has a statistically significant increase or decrease in miRNA concentration, and therefore infer whether that subject is suffering from a plasma cell disorder.

Accordingly, the inventors have realised that the difference in concentrations of miRNAs between the normal (i.e. control) and increased/decreased levels, can be used as a physiological marker, suggestive of the presence (or absence in respect of benign conditions) of a plasma cell disorder in the test subject. It will be appreciated that if a subject has an increased (or decreased) concentration of one or more signature miRNAs which is considerably higher (or lower) than the ‘normal’ concentration of that miRNA in the reference, control value, then they would be at a higher risk of having a plasma cell disorder, or a condition that was more advanced, than if the concentration of that miRNA was only marginally higher than the ‘normal’ concentration.

By way of example, the increase in concentration of miRNA compared to the ‘normal’ concentration may be at least 5-, 10, 15- or 20-fold higher than the ‘normal’ or reference concentration. By way of example, the decrease in concentration of miRNA compared to the ‘normal’ concentration may be at least 5-, 10, 15- or 20-fold lower than the ‘normal’ or reference concentration. Such changes in miRNA concentration infer that the test subject is suffering from a plasma cell disorder. Accordingly, a clinician would be able to make a decision as to the preferred course of treatment required, for example the type and dosage of the therapeutic agent according to the fifth aspect to be administered.

In the method of the second aspect and the kit of the fourth aspect, the difference in the concentration of the one or more type of microRNA molecules in the bodily sample compared to the corresponding concentration in the reference is indicative of the efficacy of treating the subject's plasma cell disorder with the therapeutic agent, for example thalidomide. The difference may be an increase or a decrease in the concentration of the one or more type of microRNA molecules in the bodily sample compared to the reference value. In embodiments where the concentration of the one or more type of microRNA molecules in the bodily sample is lower than the corresponding concentration in the reference, then this would indicate that the therapeutic agent is successfully treating the plasma cell disorder in the test subject. Conversely, where the concentration of the one or more type of microRNA molecule in the bodily sample is higher than the corresponding concentration in the reference, then this would indicate that the therapeutic agent is not successfully treating the plasma cell disorder.

It will be appreciated that the invention extends to any nucleic acid or variant, derivative or analogue thereof, which comprises substantially the nucleic acid sequences of any of the sequences referred to herein, including functional variants or functional fragments thereof. The terms “substantially the nucleotide sequence”, “functional variant” and “functional fragment”, can be a sequence that has at least 40% sequence identity with the nucleotide sequences of any one of the sequences referred to herein, for example 40% identity with the nucleotide identified as SEQ ID No:1 (i.e. miRNA-15a) or SEQ ID No.2 (i.e. miRNA-15b), and so on, for all 13 miRNAs described herein.

Nucleotide sequences with a sequence identity which is greater than 65%, more preferably greater than 70%, even more preferably greater than 75%, and still more preferably greater than 80% sequence identity to any of the sequences referred to are also envisaged. Preferably, the nucleotide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two nucleotide sequences. In order to calculate the percentage identity between two nucleotide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two nucleotide sequences may then be calculated from such an alignment as (N/T)*100, where N is the number of positions at which the sequences share an identical residue, and T is the total number of positions compared including gaps but excluding overhangs. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to the sequences shown in SEQ ID No's: 1-15, or their complements under stringent conditions. By stringent conditions, we mean the nucleotide hybridises to filter-bound DNA or RNA in 3x sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences described herein.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will known the nucleotide sequences encoding these amino acids.

According to a further aspect of the present invention, there is provided a method for diagnosing a subject suffering from a plasma cell disorder, or a pre-disposition thereto, or for providing a prognosis of the subject's condition, the method comprising analysing the concentration of one or more type of microRNA molecule in a bodily sample from a test subject and comparing this concentration with a reference for the concentration of the one or more type of microRNA molecule in an individual who does not suffer from a plasma cell disorder, wherein an increased concentration of the one or more type of microRNA molecule in the bodily sample from the test subject compared to the reference suggests that the subject is suffering from a plasma cell disorder, or has a pre-disposition thereto, or provides a negative prognosis of the subject's condition.

According to a further aspect of the invention, there is provided a kit for diagnosing a subject suffering from a plasma cell disorder, or a pre-disposition thereto, or for providing a prognosis of the subject's condition, the kit comprising:
(i) means for determining the concentration of one or more type of micro RNA molecule in a sample from a test subject; and
(ii) a reference for the concentration of the one or more type of microRNA molecule in a sample from an individual who does not suffer from a plasma cell disorder,
   wherein the kit is used to identify an increased concentration of the one or more type of microRNA molecule in the sample from the test subject compared to the reference concentration, thereby suggesting that the test subject suffers from a plasma cell disorder, or has a pre-disposition thereto, or providing a negative prognosis of the subject's condition.

According to another aspect of the invention, there is provided a method of treating an individual suffering from a plasma cell disorder, said method comprising the steps of:
(i) determining the concentration of one or more microRNA molecule in a sample having been obtained from a test subject, wherein an increased concentration of the one or more type of microRNA molecule in the bodily sample from the test subject compared to the concentration of the one or more type of microRNA molecule in a sample from an individual who does not suffer from a plasma cell disorder, suggests that the test subject suffers from a plasma cell disorder, or is pre-disposed thereto, or has a negative prognosis; and
(ii) administering, to the test subject, a therapeutic agent that prevents, reduces or delays progression of the plasma cell disorder.

It is preferred that the methods and kits may comprise analysing the concentration of one or more type of microRNA molecule selected from the group of miRNA molecules consisting of miRNA-15a, miRNA-15b, miRNA-16 and miRNA-221.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:

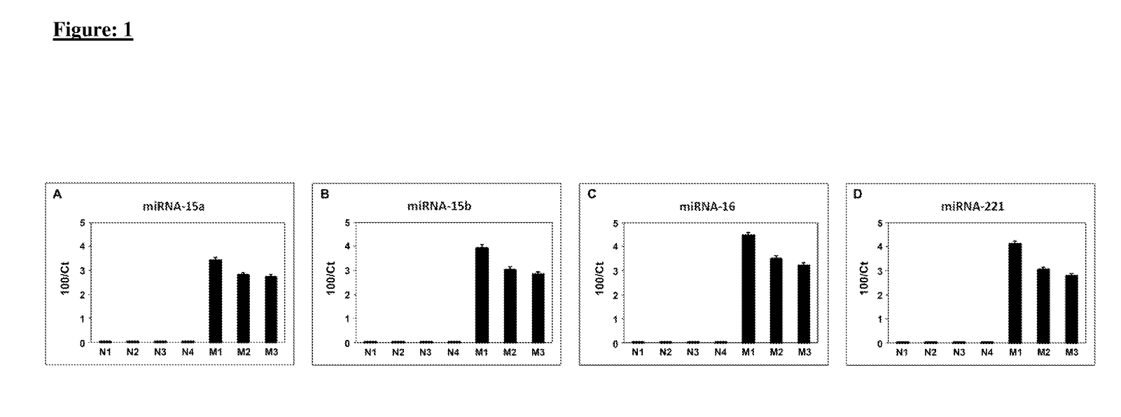

FIG. 1 shows the expression levels of four miRNAs in the blood plasma of myeloma patients compared to healthy controls. Panels A-D represent levels of miRNAs 15a (panel A), 15b (panel B), 16 (panel C) and 221 (panel D) as measured by Taqman RT-PCR. Total RNA plus miRNAs was isolated from 400 μl plasma using a mirVANA PARIS kit (Ambion) and the equivalent of 20 μl plasma used in the Taqman microRNA assay (ABI). Quantitation is expressed as 100/Ct. Four replicates were carried out for each plasma sample and the means and standard errors shown. N1-N4 are healthy subjects (N1=40 yr old male, N2=51 yr old female, N3=63 yr old female, N4=64 yr old male). M1-M3 represent myeloma patients (M1=75 yr old male, M2=44 yr old female, M3=94 yr old female);

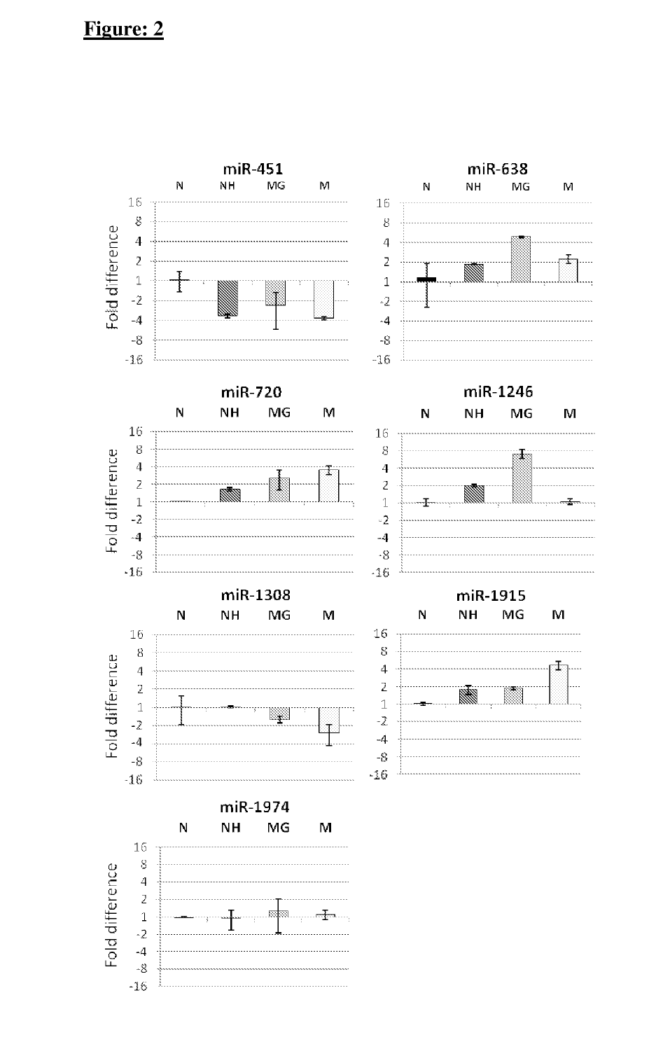

FIG. 2 shows the expression levels of seven miRNAs in the blood serum of myeloma patients compared to healthy controls. RNA was extracted in duplicates from pooled serum samples from MGUS (MG, n=20), myeloma (M, n=20) and non-myeloma hospitalised patients (NH, n=20) and from healthy controls (N, n=13). There were two RNA replicates per pool and 2 qPCRs were performed 2 RT reaction replicates (8 technical replicates per pool of serum). Error bars show standard deviation between RNA replicates; and

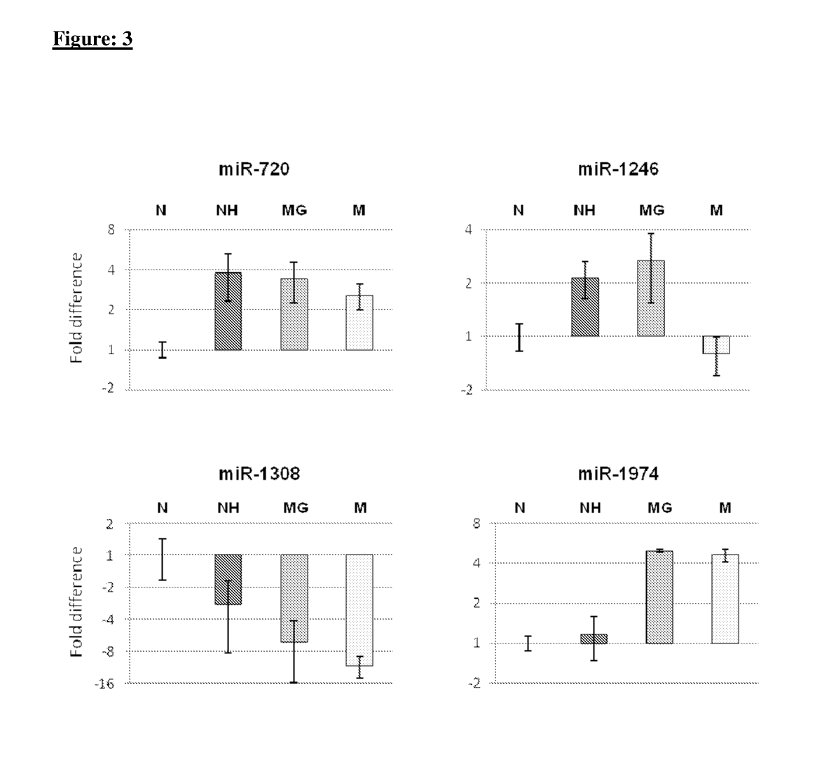

FIG. 3 shows the expression levels of four miRNAs in the blood serum of myeloma patients compared to healthy controls. RNA was extracted from individual serum samples from MGUS (MG), myeloma (M) and non-myeloma hospitalised patients (NH) and from healthy controls (N). The absolute expression levels (copy number per μl serum) of 4 miRNAs were tested in the 4 groups using specific TaqMan primers for reverse transcription and qPCR. The absolute levels were compared between groups to give a fold difference from the N group. n=6 individuals per group for miR-720, miR-1246 and miR-1308 and n=2 per group for miR-1974. For each individual/miRNA combination tested, two qPCRs were performed on 2 RT reactions (4 technical replicates). Error bars show standard error.

### EXAMPLE 1

Materials and Methods

1. Sample Collection

For this study, the inventors recruited 30 newly diagnosed myeloma patients with a target of 10 patients each of IgG, IgA and light chain only myelomas. Each of these patients were analysed at diagnosis and after completion of treatment making a total of 60 analyses. Samples were obtained from 20 MGUS patients and 20 healthy controls. This number of patients was chosen to allow for variability, but also to ensure that the amount of work involved was feasible. If any of the MGUS patients progress to the myeloma during the project, the inventors will follow the changes in their miRNA levels longitudinally.

All myeloma, MGUS and healthy controls were assessed by serum/urine electrophoresis and serum free light chain detection along with routine full blood count and biochemical profile including creatinine, calcium, albumin, LDH, CRP, β2-microglobulin, and immunoglobulin levels to assess immune paresis (Kyle, R. A. and Rajkumar, S. V. (2008) Blood, 111(6): 2962-72; McKenna, R. W., et al., Plasma cell neoplams, in Imo classification of tumours of haematopoietic and lymphoid tissues, S. H. Swerdlow, et al., Editors. 2008, International Agency for Research on Cancer: Lyon. p. 200-203). Patients with myeloma and MGUS underwent bone marrow biopsy and skeletal survey to stage their condition as per standard practice. This information was linked with the experimental results in an anonymised manner.

Clinical samples were obtained from consenting subjects under an existing research project termed “the Brighton blood disorder study” which has ethical and R&D approval for donation of blood and marrow samples for research purposes (references: 09/025/CHE and 09/H1107/1). These samples were stored under a licence from the Human Tissue

Authority and good clinical practice was adhered to at all times. Samples were collected in the clinic or phlebotomy room by trained practitioners, processed within 4 hours and transported by the named technician from the Royal Sussex County Hospital to the Medical Research Building (Sussex University campus) by car following approved Standard Operating Procedures. The samples were anonymised, coded and stored at −80° C. in freezers with an emergency generator as per Human Tissue Authority guidelines.

2. Screening of miRNAs using Whole Genome Profiling

The objective of this part of the project was to identify the particular miRNAs that are specifically up-regulated in MGUS and myeloma patients relative to healthy controls. By using microarrays, all 885 known miRNAs can be screened simultaneously to determine which ones are specifically up-regulated. Exiqon micro-array slides were used as they included almost all human miRNAs known to date (885/96.2%, miRBase version 13.0; Griffiths-Jones, S., et al. (2008) Nucleic Acids Res, 36(Database issue): D154-8) as well as 10 “spike-in” controls for normalisation. In addition, each miRNA spot was replicated 4 times and each spike-in control was replicated 48 times across the array. This microarray was used because the inventors had previously had considerable success using Exiqon arrays and could perform the experiment in-house using existing equipment. “Deep sequencing” using either Solexa (Illumina) or similar platforms were not believed to be cost-effective for this large number of samples.

3. Statistical and Bioinformatic Analysis of Microarray Data

Image analysis was conducted using the GenePixPro (7.0) software and the resulting microRNA expression data normalized using two methods, namely (i) Robust Multiarray Averaging (RMA), Irizarry, R. A., et al. (2003) Nucleic Acids Res, 31(4): e15., and (ii) GeneChip RMA (GCRMA), Wu, Z. J., et al. (2004) Journal of the American Statistical Association 99: 909-917). Differential expression analysis was conducted using the Limma package from Bioconductor that implements an Empirical Bayes approach for hypothesis testing (Smyth, G. K. (2004) Stat Appl Genet Mol Biol, 3: Article 3). The miRNAs were only defined as being differentially expressed if they met specific adjusted p-value and log odds thresholds for both normalisation methods. This resulted in a set of miRNAs which were significantly up-regulated in MGUS patients and myeloma patients compared to healthy controls. This set of miRNAs was analysed further using hierarchical clustering, to find those miRNA with related expression profiles. The functions of the up-regulated miRNA were analysed using tools such as MicroRNA and mRNA integrated analysis (MMIA), which integrates miRNA target prediction methods with functional data available in the Gene Ontology and KEGG-pathway databases (Nam, S., et al. (2009) Nucleic Acids Res, 37 (Web Server issue): W356-62). This analysis provided one (or more) sets of candidate miRNAs which were significantly and consistently up-regulated in MGUS and the three groups of myeloma patients compared to normal controls and which could be further experimentally validated by Taqman RT-PCR.

4. Confirmation of Results by Taqman Quantitative RT-PCR

From the data set produced above, the inventors confirmed and quantified the expression of four microRNAs which were significantly up-regulated according to the microarray analysis. Validation was carried out by reverse transcriptase reactions on the purified total RNA followed by TaqMan quantitative RT-PCR using primer sets specific for particular mature human miRNAs. Primer/probe sets were purchased from Applied Biosystems (http://www.appliedbiosystems.com/absite/us/en/home.html).

The reverse-transcriptase and PCR reactions were carried out as follows: An equal volume of eluted RNA (5 μl reaction=1/20 of the eluted volume) was reverse transcribed using the TagMan microRNA Reverse Transcription Kit and miRNA-specific stem-looped RT primers (Applied BioSystem) in 15 μl RT reaction (comprised of 4.16 μl of H₂O, 1.5 μl of 10× Reverse-Transcription Buffer, 0.19 μl of RNase-Inhibitor (20 units/μl), 0.15 μl of 100 mM dNTPs with dTTP, 1 μl of Multiscribe Reverse-Transcriptase, and 5 μl of input RNA) by incubating for 30 min at 16° C., 30 min at 42° C., 5 min at 85° C., and then maintained at 4° C. For real-time PCR, 1.33 μl RT products were used as templates in 20 μl reactions containing 1 μl of TagMan MicroRNA Assay (20×), 10 μl of TaqMan 2× Universal PCR Master Mix (no AmpErase UNG), and 7.67 μl of H₂O. Real-time PCR was carried out on a Mx3005P Real-Time PCR System (Stratagene) using the following conditions: 95° C. for 10 min, followed by 40 cycles at 95° C. for 15 s, and 60° C. for 1 min. All PCR reactions were run in triplicate. The threshold cycle (C_{T}) was defined as the fractional cycle number at which the fluorescence passes the fixed threshold.

The looped RT primers and probe sets, supplied by Applied Blosystems, conferred exquisite specificity for a particular miRNA. The inventors have extensive experience of using these TaqMan primer/probe sets, and the data discussed in the results section shows that they can be used on miRNAs isolated from 400 microlitres of plasma with a yield of 2.9-3.6 ng/microlitre total RNAs. The TaqMan RT-PCR not only confirmed the micro-array data but, by using spike-in controls, also provided quantitative data on the levels of miRNAs in each sample. At the end of this series of experiments, the inventors aimed to have confirmed and quantified up-regulation of at least four miRNAs which showed potential as biomarkers in plasma cell disorders.

5. Validation of miRNA Biomarkers

The next stage of the project involved choosing five of these miRNAs for each of the 4 groups and testing them on a further group of 30 myeloma patients (comprising 10 IgG, 10 IgA, and 10 light chain only myelomas), along with 10 MGUS patients and 10 healthy controls. This helped to validate the use of the four “signature” miRNAs in patient diagnoses.

6. Analysis of miRNA Biomarkers in Relation to Existing Risk Indexes and Cancer Progression

To assess the effectiveness of miRNAs as a diagnostic tool for predicting cancer progression, the inventors looked for a correlation between the expression of particular miRNAs and existing markers of disease and prognosis in MGUS and myeloma routinely performed at diagnosis and described above. Statistical considerations were undertaken to determine the appropriate sample size required to power the study in order to detect a significant association between the miRNA signature and the disease state. In addition, the inventors correlated their data with other disease-related factors including cytogenetic abnormalities, presence of end-organ failure or bony disease, age of patient, response to treatment, and overall clinical outcome.

Results

The results show that it is possible to isolate microRNAs (miRNAs) from peripheral blood plasma and accurately quantitate the levels of particular miRNAs by Taqman quantitative RT-PCR. As shown in FIG. 1, the results show that healthy control subjects express undetectable levels of the five miRNAs tested (miRNA-15a, miRNA-15b, miRNA-16, miRNA-181a, and miRNA-221). However, surprisingly, patients with myeloma express high levels of each of these miRNAs in their peripheral blood plasma with miRNA-16 being particularly highly represented (see FIG. 1).

These data therefore clearly demonstrate that it is possible to easily isolate and identify miRNAs from peripheral blood plasma, and that particular miRNAs (miRNA-15a, miRNA-15b, miRNA-16, miRNA-181a and miRNA-221) are significantly up-regulated in patients with myeloma compared to healthy controls.

### EXAMPLE 2

Based on the data generated in Example 1, the inventors have developed a diagnostic kit, which can be used for the diagnosis and prognosis of myeloma. The kit comprises a microfluidic card (or 96 well/364 well plate) pre-loaded with PCR assays, including the appropriate miRNAs that provide the “signature” for myeloma, together with relevant positive and negative controls. The signature miRNA can be any or all of the miRNAs selected from miRNA-15a, miRNA-15b, miRNA-16 and/or miRNA-221. The appropriate positive control(s) will match the signature mRNA, i.e. any or all of miRNA-15a, miRNA-15b, miRNA-16 and/or miRNA-221. The negative control is miRNA 181a and/or 181b.

miRNAs that are predictive for development of the pre-cancerous condition MGUS to myeloma can also be included as well. The practitioner, who would normally be employed in a NHS diagnostic laboratory, would isolate total RNA from plasma using a kit or machine and then apply the total RNA samples to the microfluidic card (or 96-well plate) and place it into a quantitative RT-PCR machine. The machine would then perform the required reactions (as described in Example 1) to detect the relative levels of fluorescent signal from each well of the plate. The skilled practitioner would then summarise this data and present it to the clinician to assist in his/her diagnosis.

### EXAMPLE 3

The inventors believe that the invention can be used to determine the efficacy of treating multiple myeloma with a therapeutic agent, such as cyclophoshamide, thalidomide or dexamethazone. A subject suffering from myeloma is administered with combination therapy comprising the standard treatment of cyclophosphamide, thalidomide and dexamethazone at a dose dependent upon the clinician's judgement. A blood sample is taken from the subject and then used to assess the concentration of various signature miRNAs, and these values are recorded. One month later, a second blood sample is taken from the subject, and the concentrations of the same miRNAs are determined. If the concentrations of certain miRNAs remain the same or increase over time, the clinician will realise that the treatment regime is not successful, and so can increase the dose or try alternative treatment regimes.

### EXAMPLE 4

Methods

RNA Extraction

Total RNA extraction was performed using an Ambion miRVana PARIS kit, following manufacturer's instructions. Samples were not enriched for miRNAs.

Microarrays

Agilent Human miRNA 8×15K Microarrays (V3, miRBase version 12.0) were performed following manufacturer's instructions. Hybridisation was performed using Agilent hybridization chambers in a rotary UVP HB-1000 oven. The slides were scanned using an Axon GenePix 4000B Microarray Scanner and analysed using GenePix Pro 6.0.

Quantitative PCR

Applied Biosystems TaqMan miRNA assays were used for qPCR. Reverse transcription (RT) was performed using an Applied Biosystems TaqMan MicroRNA Reverse Transcription Kit and the specific looped RT primers provided with was Taqman assay. qPCR was performed using the specific primer/probe combination provided with each TaqMan assay and Applied Biosystems TaqMan Universal PCR Master Mix, No AmpErase UNG. For absolute quantification, synthetic miRNAs from Sigma-Genesis were used for standards. Two RT reactions and two qPCRs were performed per RNA preparation from serum (four technical replicates total). One RT reaction and three qPCRs were performed for each standard (three technical replicates total).

Patient and Control Samples

The experiments described below were performed on four groups of serum samples, referred to as N, NH, MG and M. The N (“Normal”) samples were taken from normal non-hospitalised people of an age where the presence of either MGUS or myeloma conditions was unlikely (mean age 47 years). The NH (“Normal-hospitalised”) samples were taken from hospitalized non-MGUS/non-myeloma patients (mean age 77 years). The MG (MGUS) samples were taken from patients with MGUS and the M (Myeloma) samples were taken from patients with multiple myeloma. For the microarrays and initial qPCR experiments used to detect which miRNAs were expressed in each group, individual serum samples were pooled. 20 individuals were pooled for the NH, MG and M groups and 13 were pooled for the N group. RNA was extracted from the pooled serum samples in duplicate using an Ambion miRVana PARIS kit. The duplicates were referred to as N1/N2, NH1/NH2 etc. An Agilent human miRNA array was performed on each of the duplicates (eight arrays total) to detect miRNAs expressed in each group. For the miRNAs detected on the arrays, Applied Biosystems TaqMan miRNA qPCR kits were used to verify expression of each miRNA in each pool. Following this, a subset of miRNAs were chosen to be tested on RNA prepared from individual serum samples.

Results

miRNA Microarrays on Pooled Samples

Nine miRNAs were detected by the Agilent Human miRNA arrays, as shown in Table 1.

TABLE 1
miRNAs found to be expressed in MGUS/myeloma
patients and healthy controls, as detected
by Agilent Human microRNA Microarrays.

| miRNA | Sequence |
|---|---|
| hsa-miR-451 | AAACCGUUACCAUUACUGAGUU |
| hsa-miR-638 | AGGGAUCGCGGGCGGGUGGCGGCCU |
| hsa-miR-720 | UCUCGCUGGGGCCUCCA |
| hsa-miR-1246 | AAUGGAUUUUUGGAGCAGG |
| hsa-miR-1308 | GCAUGGGUGGUUCAGUGG |
| hsa-miR-1915 | CCCCAGGGCGACGCGGCGGG |
| hsa-miR-1974 | UGGUUGUAGUCCGUGCGAGAAUA |
| hsa-miR-574-5p | UGAGUGUGUGUGUGUGAGUGUGU |
| hsa-miR-762 | GGGGCUGGGGCCGGGGCCGAGC |

Each was detected in at least two replicates. Each array contained 16 spots per miRNA, and a miRNA was taken to have been detected if more than four of its spots were visible above the background level. The levels of these miRNAs were not compared between groups using the array data as there was no known internal control for use as a normaliser to compensate for technical variations between arrays.

qPCR on Pooled Samples

The main aim of this experiment was to validate the microarray results and confirm that the pattern of miRNA expression was similar using a different and more quantitative technique. TaqMan qPCR assays were available for seven of the nine miRNA detected on the arrays. These seven assays were tested on each of the replicated RNA samples (i.e. N1 /N2 etc) from pooled serum used on the arrays. Two reverse-transcriptase replicates and two PCR replicates were carried out for each pooled sample giving eight replicates for each pool in total. FIG. 2 shows that all seven miRNAs were detected in both replicates of each group and that the expression pattern of miRNAs is different for each patient group. The relative expression levels of 7 miRNAs were tested in the 4 sets of duplicates using specific TaqMan primers for reverse transcription and qPCR. The inventors were surprised to see that the expression levels of some miRNAs were increased in M and/or MGUS patients (miR-638, miR-720, miR-1246 and miR-1915) and that the levels were reduced for other miRNAs (miR-451 and miR-1308). These results mean that that these miRNA can act as robust biomarkers for different disease conditions. Although accurate quantification of expression differences between samples is not possible due to the lack of a normaliser miRNA, it seems likely that these differences reflect true biological differences as the technical variation between qPCR tests is minimal.

Absolute qPCR on Individual Samples

To determine accurate expression differences between groups, in the absence of a normaliser miRNA, absolute quantification was performed to determine the copy number of each miRNA per μl of serum. As consistent differences between individuals of the same group are a requirement, the inventors have begun a larger scale analysis of absolute miRNA levels for four miRNAs. The four miRNAs initially chosen were miR-720, miR-1246, miR-1308 and miR-1974. RNA was prepared individually from 200 μl of serum from the patients that had formed the pools for earlier experiments. Absolute quantification requires standard samples to be run alongside unknown samples for comparison. The standards used must be the miRNA of interest at a known concentration, for example, miRNAs produced synthetically. The synthetic miRNA is diluted across a range of several orders of magnitude and these dilutions are treated in the same way as the unknown samples (RT step followed by qPCR). The standard samples are used to plot a curve of cycle threshold (Ct) vs copy number and the Ct of the unknown samples is compared to this to give a copy number. The Copy numbers from individuals can then be compared between groups to show differences in expression.

FIG. 3 shows the results to date using six individuals per group (total 24) for miR-720, miR-1246 and miR-1308 (four technical replicates per individual/miRNA combination). As expected, the pattern of expression of miR-720, miR-1245 and miR-1308 was similar using individual samples compared to using pooled samples. Just two individuals per group have been tested for miR-1974 so far.

### CONCLUSION

The inventors have shown, using both microarrays and TaqMan RT-PCR, that six miRNAs (miR-451, miR-638, miR-720, miR-1246, miR-1308 and miR-1915) are expressed in specific expression patterns in serum derived from patient groups compared to controls. For three of these miRNAs (miR-720, miR-1246 and miR-1308), these different expression patterns have been confirmed in individual patients. The specific patterns seen vary between myeloma, MGUS and control patients, showing that they can be used as biomarkers for prognostic and diagnostic purposes.

1-28. (canceled)

29. A method for diagnosing a subject suffering from a plasma cell disorder, or a pre-disposition thereto, or for providing a prognosis of the subject's condition, the method comprising analysing the concentration of one or more type of microRNA molecule in a bodily sample from a test subject and comparing this concentration with a reference for the concentration of the one or more type of microRNA molecule in an individual who does not suffer from a plasma cell disorder, wherein a difference in the concentration of the one or more type of microRNA molecule in the bodily sample from the test subject compared to the reference suggests that the subject is suffering from a plasma cell disorder, or has a pre-disposition thereto, or provides a negative prognosis of the subject's condition.

30. A method according to claim 29, wherein the plasma cell disorder is amyloidosis, Waldenstrom's macroglobulinemia, osteosclerotic myeloma (POEMS syndrome), Monoclonal gammopathy of unknown significance (MGUS) or plasma cell myeloma.

31. A method according to claim 29, wherein the plasma cell disorder is plasma cell myeloma.

32. A method according to claim 29, wherein the method comprises analysing the concentration of one or more type of microRNA molecule selected from the group of miRNA molecules consisting of miRNA-15a, miRNA-15b, miRNA-16, miRNA-221, miRNA-451, miRNA-638, miRNA-720, miRNA-1246, miRNA-1308, miRNA-1915, miRNA-1974, miRNA-574-5p and miRNA-762.

33. A method according to claim 29, wherein the method comprises analysing the concentration of one or more type of microRNA molecule selected from the group of miRNA molecules consisting of miRNA-638, miRNA-720, miRNA-1246, miRNA-1308 and miRNA-1915.

34. A method according to claim 29, wherein the difference in concentration is an increase compared to the reference, and wherein the method comprises analysing the concentration of one or more type of microRNA molecule selected from the group of miRNA molecules consisting of miRNA-15a, miRNA-15b, miRNA-16, miRNA-221, miRNA-638, miRNA-720, miRNA-1246 and miRNA-1915.

35. A method according to claim 34, wherein the increase in concentration of miRNA compared to the reference concentration is at least 5-, 10, 15- or 20-fold higher than the reference concentration.

36. A method according to claim 29, wherein the difference in concentration is a decrease compared to the reference, and wherein the method comprises analysing the concentration of miRNA-451 and/or miRNA 1308.

37. A method according to claim 36, wherein the decrease in concentration of miRNA compared to the reference concentration is at least 5-, 10, 15- or 20-fold lower than the reference concentration.

38. A method according to claim 29, wherein the method comprises determining the concentration of one or more type of microRNA molecule selected from the group of miRNA molecules comprising a nucleotide sequence substantially as set out in any or SEQ ID No.1 to 13, or any of the complementary sequences thereof, or variants or fragments thereof.

39. A method according to claim 29, wherein the bodily sample is a blood plasma sample or a blood serum sample.

40. A kit for diagnosing a subject suffering from a plasma cell disorder, or a pre-disposition thereto, or for providing a prognosis of the subject's condition, the kit comprising:
(i) means for determining the concentration of one or more type of micro RNA molecule in a sample from a test subject; and
(ii) a reference for the concentration of the one or more type of microRNA molecule in a sample from an individual who does not suffer from a plasma cell disorder,
wherein the kit is used to identify a difference in the concentration of the one or more type of microRNA molecule in the sample from the test subject compared to the reference concentration, thereby suggesting that the test subject suffers from a plasma cell disorder, or has a pre-disposition thereto, or providing a negative prognosis of the subject's condition.

41. A kit according to claim 40, wherein the kit is for carrying out a method for diagnosing a subject suffering from a plasma cell disorder, or a pre-disposition thereto, or for providing a prognosis of the subject's condition, the method comprising analysing the concentration of one or more type of microRNA molecule in a bodily sample from a test subject and comparing this concentration with a reference for the concentration of the one or more type of microRNA molecule in an individual who does not suffer from a plasma cell disorder, wherein a difference in the concentration of the one or more type of microRNA molecule in the bodily sample from the test subject compared to the reference suggests that the subject is suffering from a plasma cell disorder, or has a pre-disposition thereto, or provides a negative prognosis of the subject's condition.

42. A kit according to claim 40, wherein the kit comprises detection means for determining the concentration of the one or more type of miRNA in the sample once this has been obtained from the subject.

43. A kit according to claim 42, wherein the detection means comprises one or more primer, for use in a PCR method for amplifying the miRNA.

44. A kit according to claim 42, wherein the kit comprises a positive control, which corresponds to total RNA extracted from a sample of a subject having a plasma cell disorder where it has been established that the relevant miRNAs are present at statistically higher levels than normal controls, wherein the positive control comprises a nucleotide sequence substantially as set out in SEQ ID No.1-13, or the complementary sequence thereof, or a variant, or fragment thereof

45. A kit according to claim 42, wherein the kit comprises a negative control, which corresponds to total RNA extracted from a sample of a normal, healthy subject without a plasma cell disorder, where it has previously been established that the relevant miRNAs are not detectable, wherein the negative control comprises a nucleotide sequence substantially as set out in SEQ ID No.14 or 15, or the complementary sequence thereof, or a variant or fragment thereof.

46. A method of treating an individual suffering from a plasma cell disorder, said method comprising the steps of:
(i) determining the concentration of one or more microRNA molecule in a sample having been obtained from a test subject, wherein a difference in the concentration of the one or more type of microRNA molecule in the bodily sample from the test subject compared to the concentration of the one or more type of microRNA molecule in a sample from an individual who does not suffer from a plasma cell disorder, suggests that the test subject suffers from a plasma cell disorder, or is pre-disposed thereto, or has a negative prognosis; and
(ii) administering, to the test subject, a therapeutic agent that prevents, reduces or delays progression of the plasma cell disorder.

47. A method for determining the efficacy of treating a subject suffering from a plasma cell disorder with a therapeutic agent, the method comprising analysing the concentration of one or more type of microRNA molecule in a bodily sample from a test subject and comparing this concentration with a reference for the concentration of the one or more type of microRNA molecule in an individual who does not suffer from a plasma cell disorder, wherein a difference in the concentration of the one or more type of microRNA molecule in the bodily sample compared to the reference is indicative of the efficacy of treating the test subject with the therapeutic agent.

48. A kit for determining the efficacy of treating a subject suffering from a plasma cell disorder with a therapeutic agent, the kit comprising:
(i) means for determining the concentration of one or more type of microRNA molecule in a sample from a test subject; and
(ii) a reference for the concentration of the one or more type of microRNA molecule in a sample from an individual who does not suffer from a plasma cell disorder,
wherein the kit is used to identify a difference in the concentration of the one or more type of microRNA molecule in the sample from the test subject compared to the reference concentration, the difference in concentration being indicative of the efficacy of treating the test subject with the therapeutic agent.
